(19) United States
(12) Patent Application Publication
(10) Pub. No.: US 2024/0341627 A1
(43) Pub. Date: Oct. 17, 2024

(54) PROTECTED SAMPLE COLLECTION MEDIA

(71) Applicant: 3M INNOVATIVE PROPERTIES COMPANY, St. Paul, MN (US)

(72) Inventors: Audrey A. Sherman, Woodbury, MN (US); Alan R. Dombrowski, Woodbury, MN (US); Michael R. Berrigan, Oakdake, MN (US); Brett J. Sitter, Marine on St Croix, MN (US); Tonya D. Bonilla, Woodbury, MN (US); Narina Y. Stepanova, Woodbury, MN (US); Laura R. Nereng, Woodbury, MN (US); Michael C. Wohl, Bethesda, MD (US)

(21) Appl. No.: 18/580,284

(22) PCT Filed: Jul. 20, 2022
(86) PCT No.: PCT/IB2022/056708

§ 371 Date: Jan. 18, 2024

Related U.S. Application Data
(60) Provisional application No. 63/203,441, filed on Jul. 22, 2021.

(51) Int. Cl. A61B 5/097; A61B 5/08; A61B 10/00

(52) U.S. Cl. CPC A61B 5/097; A61B 5/082; A61B 10/0096; A61B 2010/0087

(57) ABSTRACT

A protected sample collection media for use in a sample collection device includes a piece of porous sample collection media having a first major side and an opposing second major side; and a protective sheath fully covering the first major side and the second major side. The protective sheath has a first portion and a second portion. The first portion is removable without removing the second portion. The second portion forms a tab constructed to be gripped by hand. The protected sample collection media may be part of a system including a sample collection device. The system may be provided as a kit including instructions for use of the kit.

### FIELD

The present disclosure relates to sample collection media for use in sample collection devices and systems. The present disclosure relates to sample collection media protected by a removable sheath.

### BACKGROUND

Diagnostic tests used to test for the presence of a virus or other pathogen in the airways, throat, or nasopharynx typically involve the insertion of a swab into the back of the nasal passage, the mid-turbinate area of the nasal passage, the anterior nares, or the throat to obtain a sample. The swab is then inserted into a container and analyzed or sent to a lab for processing. Other diagnostic tests involve collecting a saliva sample and then placing it in a container. Currently available at-home viral tests (e.g., COVID-19 tests) involve a nasal swab and a test kit (for example, the Ellume™ test, the Abbot™ BinaxNOW™ test, and the Lucira™ All-in-One test kit). Tests that utilize nasal swab samples or saliva contend with contaminants that can interfere with the various diagnostic tests. As a result, these sample types require a purification step when using RT-PCR molecular testing.

### SUMMARY

There is a need for inexpensive, simple to use, and reliable sample collection devices and systems that may be used by laypeople to obtain a sample for testing for the presence of a target virus, target pathogen, or other target analyte, in a collected sample. There is further a need for sample collection media that can be conveniently and easily stored, transported, and handled while minimizing contamination from the environment or from a user, for use in such devices and systems.

The protected sample collection media of the present disclosure is suitable for use in various sample collection devices and systems. The sample collection devices may include a housing that receives the sample collection media and facilitates depositing a sample (e.g., a bioaerosol, such as an exhalation airflow sample) onto the sample collection media. The housing defines an airflow path and the sample collection media received in the housing may be arranged to occlude the airflow path such that viruses, bacteria, or other pathogens in the airflow are captured by the sample collection media. A sample collection system may include a sample collection device and one or more other implements to facilitate analysis of the sample. The sample collection system may include a sample collection tube, an assay, or both.

It is desirable to provide sample collection media that is easy to use while protected from contamination. The protected sample collection media may advantageously be self-contained and optionally sterile. A self-contained (and optionally sterile) sample collection media may improve accuracy and reliability of pathogen testing due to the reduced contamination and background noise, unlike media or swabs exposed to the environment and other test collection devices which may be contaminated upon use and/or during testing.

It is further desirable to provide sample collection media which, after sample collection, provides for easy transfer of the collected sample to a sample collection tube or assay without contamination from the user or the environment.

According to an embodiment, a protected sample collection media for use in a sample collection device includes a piece of porous sample collection media having a first major side and an opposing second major side; and a protective sheath fully covering the first major side and the second major side. The protective sheath has a first portion and a second portion. The first portion is removable without removing the second portion. The second portion forms a tab constructed to be gripped by hand.

The protective sheath may include porous paper, plastic, metal, nonwoven sheet, or a combination thereof.

In some embodiments, the first portion of the protective sheath comprises a peelable first side disposed on the first major side of the piece of porous sample collection media and a peelable second side disposed on the first major side of the piece of porous sample collection media.

In some embodiments, the first and second portions are integral with one another. A separation between the first and second portions may be marked by notches or perforations.

The porous sample collection media may be made of a nonwoven material. The nonwoven material may include polylactic acid, polypropylene, or a combination thereof.

The protected sample collection media may further include a supportive frame disposed along the protective sheath or piece of porous sample collection media. The supportive frame may extend along outer edges of the protective sheath or piece of porous sample collection media.

According to an embodiment, a sample collection system includes the protected sample collection media and a sample collection device. The sample collection device has an airflow path extending from a first end to a second end, and an opening constructed to receive the protected sample collection media such that the sample collection media occludes the airflow path. The tab may at least partially extend outside of the sample collection device. The sample collection device may have a tubular wall defining the first end and the second end, and wherein the first end forms a mouthpiece. The opening may be disposed in the tubular wall. The tubular wall may have a track on an inside surface of the tubular wall constructed to guide or support the protected sample collection media. The sample collection device may be constructed for coupling with a sample collection tube via a bayonet coupling, interference fit, snap fit, or threaded coupling.

According to an embodiment, a kit for collecting a sample includes the sample collection system and instructions to: grip the protected sample collection media by the second portion; insert the protected sample collection media into the sample collection device; exhale along the airflow path to capture a sample in the porous sample collection media; remove the protected sample collection media from the sample collection device; and remove the first portion of the protective sheath. The kit may further include instructions to transfer the loaded sample collection media into a sample collection tube. The kit may further include instructions to elute the sample from the loaded sample collection media using a liquid.

### BRIEF DESCRIPTION OF FIGURES

FIG. 1A is a schematic top view of protected sample collection media according to an embodiment.

FIG. 1B is a schematic top view of the sample collection media of FIG. 1A with a first portion of the protective sheath removed.

FIG. 2 is a perspective view of protected sample collection media with a first portion of the protective sheath partially peeled off according to an embodiment.

FIG. 3A is a schematic top view of sample collection media with a supportive frame according to an embodiment.

FIG. 3B is a schematic perspective view of sample collection media with a supportive frame according to an embodiment.

FIG. 4A is a perspective view of the sample collection media of FIG. 3A placed in a sample collection device according to an embodiment.

FIG. 4B is a bottom perspective view of the sample collection media and sample collection device of FIG. 4A.

FIG. 4C is a cross-sectional view of the sample collection media and sample collection device of FIG. 4A.

FIG. 4D is a partial cut-off view of the sample collection media and sample collection device of FIG. 4A.

FIG. 4E is a perspective view of the sample collection media and sample collection device of FIG. 4A with a sample collection tube.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer” and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

The terms “downstream” and “upstream” refer to a relative position based on a direction of exhalation airflow through the device. For example, the upstream-most element of the device is the mouthpiece element, and the downstream-most element of the device is the exhalation outlet element.

All headings provided herein are for the convenience of the reader and should not be used to limit the meaning of any text that follows the heading, unless so specified.

The term “i.e.” is used here as an abbreviation for the Latin phrase id est, and means “that is,” while “e.g.” is used as an abbreviation for the Latin phrase exempli gratia and means “for example.”

All scientific and technical terms used herein have meanings commonly used in the art unless otherwise specified. The definitions provided herein are to facilitate understanding of certain terms used frequently herein and are not meant to limit the scope of the present disclosure.

The term “about” is used here in conjunction with numeric values to include normal variations in measurements as expected by persons skilled in the art and is understood have the same meaning as “approximately” and to cover a typical margin of error, such as +5% of the stated value. Moreover, unless otherwise indicated, all numbers expressing quantities, and all terms expressing direction/orientation (e.g., vertical, horizontal, parallel, perpendicular, etc.) in the specification and claims are to be understood as being modified in all instances by the term “about.”

Terms such as “a,” “an,” and “the” are not intended to refer to only a singular entity but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range. As used here, “have”, “having”, “include”, “including”, “comprise”, “comprising” or the like are used in their open-ended sense, and generally mean “including, but not limited to.” It will be understood that “consisting essentially of.” “consisting of,” and the like are subsumed in “comprising” and the like. As used herein, “consisting essentially of,” as it relates to a composition, product, method or the like, means that the components of the composition, product, method or the like are limited to the enumerated components and any other components that do not materially affect the basic and novel characteristic(s) of the composition, product, method or the like.

The term “substantially” as used here has the same meaning as “significantly,” and can be understood to modify the term that follows by at least about 90%, at least about 95%, or at least about 98%. The term “not substantially” as used here has the same meaning as “not significantly,” and can be understood to have the inverse meaning of “substantially,” i.e., modifying the term that follows by not more than 10%, not more than 5%, or not more than 2%.

The words “preferred” and “preferably” refer to embodiments that may afford certain benefits. under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Any direction referred to here, such as “front,” “back,” “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

### DETAILED DESCRIPTION

The present disclosure relates to sample collection media for use in sample collection devices and systems. The present disclosure relates to sample collection media protected by a removable sheath. The protected sample collection media may be used in devices and systems for collecting bioaerosol samples.

Generally, the sample collection media may be used in a sample collection device that includes a housing constructed to receive the sample collection media. The housing defines an airflow path and the protected sample collection media received in the housing is arranged to occlude the airflow path such that viruses, bacteria, or other pathogens in the airflow are captured by the sample collection media. The sample collection media may include porous nonwoven material constructed to capture viruses. pathogens, or other analytes, carried in an exhalation airflow.

After loading, the sample collection media may be transferred into a sample collection tube. The protective sheath may be removed prior to transferring the sample into the sample collection tube. The protective sheath may be configured such that the protective sheath may be removed and the sample collection media may be transferred into the sample collection tube without touching the sample collection media with a user's hands. The sample may further be analyzed for the presence of a pathogen of interest, or the sample collection tube may be capped and sent away for analysis. According to an alternative embodiment, the sample may be transferred into the sample collection tube by passing a liquid through the porous sample collection media to elute the sample, including pathogens, viruses, or other analytes, bound to the porous sample collection media, forming an eluate, and allowing the eluate to flow into the sample collection tube. The eluate may then be analyzed using known methods.

According to an embodiment, the protected sample collection media is easy to use while protecting the sample collection media from contamination. The protected sample collection media may advantageously be self-contained and optionally sterile. The protected sample collection media may improve accuracy and reliability of pathogen testing due to the reduced contamination and background noise. The protected sample collection media provides for easy transfer of the collected sample to a sample collection tube or assay while reducing contamination from the user or the environment.

According to an embodiment, the protected sample collection media includes a piece of porous sample collection media covered by a removable protective sheath. The porous sample collection media may be a flat sheet, having a first major side and an opposing second major side. The protective sheath fully covers the first major side and the second major side. The protective sheath has a first portion and a second portion, where the first portion is removable without removing the second portion. The second portion forms a tab constructed to be gripped by hand. The second portion covers only a part of the porous sample collection media. For example, the second portion may cover 50% or less, 40% or less. 30% or less, or 20% or less of the length of the porous sample collection media.

The protective sheath may be made of a single sheet of material that is wrapped on both major sides of the porous sample collection media. The protective sheath may be made of two sheets of material that are applied on the porous sample collection media and adhered together along the edges. The protective sheath may be made of more than two sheets of material to provide the first portion and the second portion.

In some embodiments, the first and second portions of the protective sheath are integral with one another. In such embodiments, the first portion may be removed by tearing. A separation between the first and second portions may be marked by notches or perforations. A user may grip the protected sample collection media by the second part and tear off the first portion along the notches or perforations, leaving the gripped end of the porous sample collection media covered by the second part.

In some embodiments, the first portion of the protective sheath has a peelable first side disposed on the first major side of the piece of porous sample collection media and a peelable second side disposed on the second major side of the piece of porous sample collection media. A user may remove the first portion by peeling off the first side and then removing or peeling off the second side, leaving the second portion in place.

The protective sheath may be made of any suitable material. For example, the protective sheath may be made of paper, plastic. metal, nonwoven sheet, or a combination thereof. The protective sheath may be made of a porous laminated or coated material, such as plasticized paper, metalized paper.

metalized plastic, and the like. In some embodiments, the protective sheath may be made of nonwoven material. The nonwoven material may include polylactic acid, polypropylene, or a combination thereof. The nonwoven material may have a suitable thickness and strength that provides protection for the porous sample collection media before removal of the first portion but also allows easy removal of the first portion, for example, by tearing.

The protective sheath may act as a pre-filter or screen covering the porous sample collection media during sample taking. The protective sheath may catch larger particles (larger than viruses or pathogens) and prevent such particles from reaching the porous sample collection media. The exhalation airflow passes through a thickness of the protective sheath. In some cases, the protective sheath may be a non-woven layer that does not have an electrostatic charge. In some embodiments, the protective sheath does not capture significant amounts of viral material, pathogen material, or other analyte material, and instead allows them to transmit through the protective sheath. According to an embodiment, the protective sheath is removed from the porous sample collection media before transferring the porous sample collection media to a sample collection tube or test tube for analysis.

The porous sample collection media may be provided in any shape and size depending on its intended use. In some embodiments, the porous sample collection media is made of a planar sheet of material. However, other configurations are also contemplated, as long as the porous sample collection media is capable of being protected by a protective sheath as described, and capable of being inserted into a sample collection device. While the porous sample collection media is illustrated here as rectangular or substantially rectangular planar clement, it is understood that the porous sample collection media may define any suitable shape for being disposed within the housing and along the airflow path. The shape and size of the porous sample collection media may be selected based on the shape and size of the interior of the sample collection device.

The porous sample collection media may be formed of any suitable material that is capable of capturing viruses, pathogens, or other analytes from exhalation airflow and releasing the captured viruses. pathogens, or other analytes upon being contacted with an eluent, such as a saline solution. The porous sample collection media may be formed of polymeric material. The porous sample collection media may be formed of a polyolefin. Examples of suitable polyolefins include polypropylene, polylactic acid, and the like, and a combination thereof. In one embodiment the porous sample collection media is formed of polypropylene. In one embodiment the porous sample collection media is formed of polylactic acid. One illustrative porous sample collection media is commercially available from 3M Company (St. Paul MN. U.S.A.) under the trade designation FILTRETE Smart MPR 1900 Premium Allergen, Bacteria & Virus Air Filter Merv 13.

The porous sample collection media may be a nonwoven material capable of capturing pathogens, viruses, or other analytes from an exhalation airflow. According to an embodiment, the porous sample collection media is a nonwoven material carrying an electrostatic charge. The electrostatic charge may enable capturing pathogens, viruses, or other analytes from an exhalation airflow. In some cases, the porous sample collection media may be a hydrophobic nonwoven material. In other cases, the porous sample collection media may be a hydrophilic nonwoven material. The porous sample collection media may be a hydrophobic nonwoven material carrying an electrostatic charge configured to capture pathogens, viruses, or other analytes from an exhalation airflow. The porous sample collection media may be a hydrophilic nonwoven material carrying an electrostatic charge configured to capture pathogens. viruses, or other analytes from an exhalation airflow. The term “hydrophobic” refers to a material having a water contact angle of 90 degrees or greater, or from about 90 degrees to about 170 degrees, or from about 100 degrees to about 150 degrees. The term “hydrophilic” refers to a material having a water contact angle of less than 90 degrees. Water contact angle is measured using ASTM D5727-1997 Standard test method for surface wettability and absorbency of sheeted material using an automated contact angle tester.

The porous sample collection media may have a thickness (orthogonal to the major plane) of 200 μm or greater or 250 μm or greater. The porous sample collection media may have a thickness of 750 μm or less or 1000 μm or less. The porous sample collection media may have a thickness of in a range from 200 μm to 1000 μm, or from 250 μm to 750 μm. The porous sample collection media may have major plane surface area (of one side) of 1 cm² or greater or 2 cm² or greater. The porous sample collection media may have major plane surface area of 3 cm² or less or 4 cm² or less. The porous sample collection media may have major plane surface area in a range from 1 cm² to 4 cm², or 2 cm² to 3 cm².

In some embodiments, the protected sample collection media is supported by a frame. A frame may facilitate insertion of the protected sample collection media into a sample collection device. The supportive frame may be disposed along the protected sample collection media. The supportive frame may extend along outer edges of the protective sheath or the piece of porous sample collection media. The supportive frame may be adhered to the protective sheath or the porous sample collection media by an adhesive. According to an embodiment, the supportive frame is adhered to the protective sheath.

The protected sample collection media is configured for use in a sample collection device. For example, the protected sample collection media may be configured for use in a sample collection device described in U.S. Provisional Patent Application No. 63/200,058; U.S. Provisional Patent Application No. 63/200,901; U.S. Provisional Patent Application No. 63/202,140; U.S. Provisional Patent Application No. 63/200,958; U.S. Provisional Patent Application No. 63/201,981; U.S. Provisional Patent Application No. 63/202,143; U.S. Provisional Patent Application No. 63/201,983; or U.S. Provisional Patent Application No. 63/203,442 filed concurrently with the present disclosure. Generally, the protected sample collection media is configured for use in a sample collection device that has a housing defining an airflow path. The protected sample collection media may be placed (e.g., inserted) within the housing and arranged to occlude the airflow path. The housing may be modified to include an opening for inserting the protected sample collection media. The user may exhale into the sample collection device and load the porous sample collection media with a sample of the exhalation airflow to form a loaded porous sample collection media. The user may exhale through an opening, e.g., through a mouthpiece. The housing is constructed so that by exhaling through the opening in the mouthpiece, at least a portion of the exhalation airflow passes through the porous sample collection media. The user may optionally transfer the loaded sample collection media into a sample collection tube. Alternatively, the user may elute the sample from the loaded sample collection media directly into the sample collection tube or onto an assay.

In some embodiments, the protected sample collection device is configured for insertion of the porous sample collection media into the sample collection device and into the airflow path. The sample collection device may have a housing defining an airflow path extending from a first end to a second end. and an opening constructed to receive the protected sample collection media such that the porous sample collection media occludes the airflow path. A user may insert the protected sample collection media (porous sample collection media covered by the protective sheath) into the airflow path through the opening. When the protected sample collection media is inserted into the sample collection device, a part of the protected sample collection media (e.g., a tab) extends outside of the sample collection device. The sample collection device may include various features to facilitate the insertion of the protected sample collection media, as further discussed below. The user may breathe through the sample collection device to capture a sample of exhalation breath in the porous sample collection media. The protected sample collection media may then be removed from the sample collection device. The protected sample collection media may be gripped by the tab (the second portion of the protective sheath). The first portion of the protective sheath may be removed (e.g., by tearing or peeling), leaving the second portion to cover a part of the sample collection media (e.g., the tab). In some embodiments, the first portion of the protective sheath may remain in the sample collection device. The loaded sample collection media may then be transferred into a sample collection tube.

The protected sample collection media and the sample collection device may be provided as a complete system for collecting a sample from exhalation breath. The system may further include other parts, such as a sample collection tube for receiving the loaded sample collection media, or an assay for testing the loaded sample collection media for the presence of a pathogen or other analyte.

According to an embodiment, the housing has a proximal end and an opposing distal end. The distal end may be constructed to couple with a sample collection tube. The distal end may include any suitable mechanism for coupling with a sample collection tube. For example, the distal end may by constructed for bayonet coupling, interference fit, snap fit, or threaded coupling. Many commercially available sample collection tubes or test tubes have a threaded top for attaching a cap. The distal end of the sample collection device may be constructed to couple with the threads of the sample collection tube. The distal end of the sample collection device may include internal threading configured to couple with the external threading of the sample collection tube. In some embodiments, the distal end is configured for an interference fit with a sample collection tube.

The loaded sample collection media may be transferred into the sample collection tube by removing the protected sample collection media from the sample collection device, removing the protective sheath (e.g., the first portion or both the first and second portions), and dropping the loaded sample collection media into the tube.

The loaded sample collection media may be transferred into the sample collection tube by pushing it into the tube with a plunger while the sample collection tube is coupled with the sample collection device. In some embodiments, the plunger is a rod configured to fit through the airflow channel.

In some embodiments, the sample is eluted from the sample collection media by dispensing a liquid onto the loaded sample collection media. The liquid dispensed onto the sample collection media may be an aqueous liquid. The liquid may be a buffer solution. The liquid may be an aqueous buffer solution. The liquid may be a saline solution. The liquid may include a surfactant. The liquid may have a contact angle of greater than 90 degrees when measured on the porous sample collection media. The liquid may be a saline solution including a surfactant. The liquid (e.g., a buffer or a saline solution) may include from 0.1 wt-% or more or 0.5 wt-% or more, and up to 1 wt-% or up to 2 wt-% of surfactant. If provided as a metered dose, the liquid may have a volume of 50 μL to 500 μL.

The liquid may be applied onto the loaded porous sample collection media. The liquid may travel through the surface and thickness of the loaded porous sample collection media and flow off of the porous sample collection media carrying any virus, pathogen, or other analyte, that was present on the loaded porous sample collection media. This loaded liquid may be collected and tested.

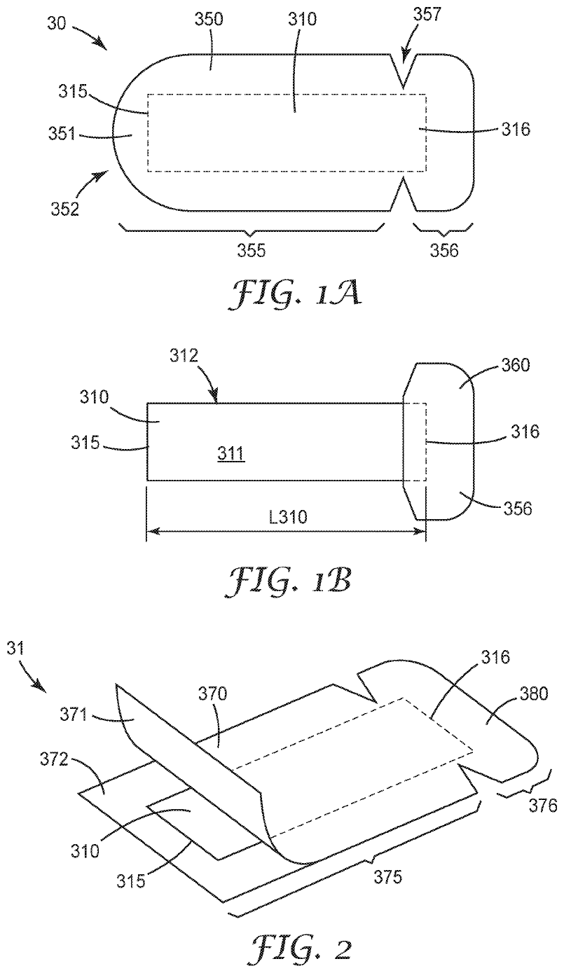

Referring now to FIGS. 1A and 1B, a schematic depiction of a protected sample collection media 30 is shown. The protected sample collection media 30 includes a piece of sample collection media 310 protected by a removable sheath 350. The sample collection media 310 may be in the form of a rectangular strip of material, as shown, or any other suitable shape as desired. The depicted sample collection media 310 has a first major side 311 and an opposing second major side 312. The protective sheath 350 fully covers the first major side 311 and the second major side 312. The protective sheath 350 includes a first side 351 that covers the first side 311 of the sample collection media 310 and a second side 352 that covers the second side 312 of the sample collection media 310.

The sample collection media 310 has a first end 315 and an opposite second end 316. The protective sheath 350 defines a first portion 355 and a second portion 356. The first portion 355 of the protective sheath 350 covers the first end 315 of the sample collection media and the second portion 356 covers the second end 316. According to an embodiment, the first portion 355 is removable without removing the second portion 356. The second portion 356 may further be removed if desired.

The first and second portions 355. 356 of the protective sheath 350 may be integral with one another. The sheath 350 may include notches 357 between the first and second portions 355. 356. Alternatively, the sheath 350 may include perforations between the first and second portions 355. 356. A user may grip the protected sample collection media 30 by the second portion 356 and tear off the first portion 355 along the notches (or perforations), leaving the second end 316 of the porous sample collection media 310 covered by the second portion 356.

The second portion 356 forms a tab 360 constructed to be gripped by hand. The second portion 356 covers only a part of the sample collection media 310. For example, the second portion 356 may cover 50% or less, 40% or less, 30% or less, or 20% or less of the length L310 of the sample collection media 310.

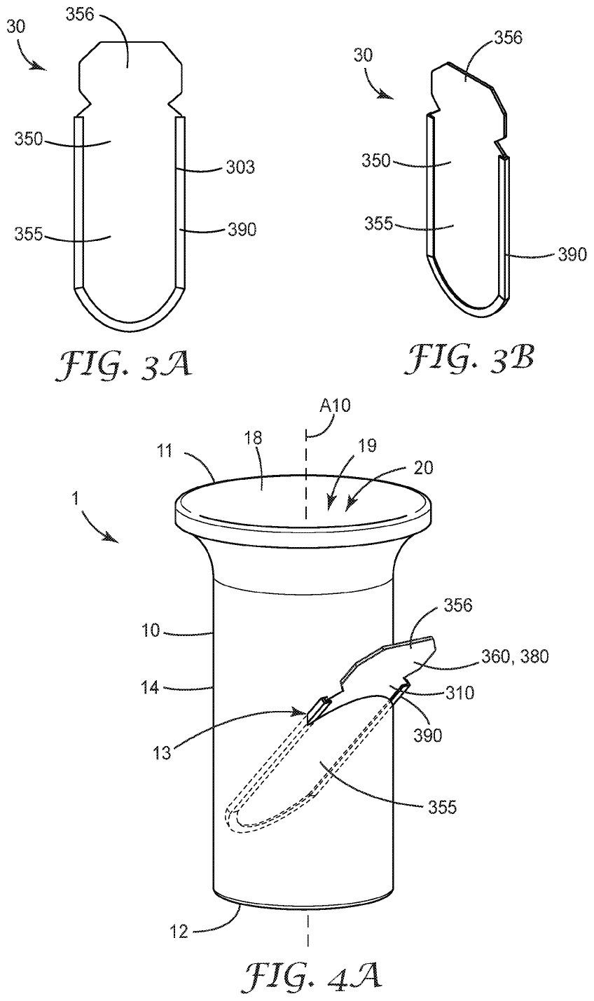

In an alternative embodiment, the sample collection media 310 is protected by a peelable sheath 370, as shown in FIG. 2. The peelable sheath 370 includes a first portion 375 and second portion 376. The first portion 375 has a peelable first side 371 disposed on the first major side 311 of the sample collection media 310 and a peelable second 372 side disposed on the second major side 312 of the sample collection media 310. A user may remove the first portion 375 by peeling off the first side 371 and then removing or peeling off the second side 372, leaving the second portion 376 in place. The second portion 376 forms a tab 380 constructed to be gripped by hand. The second portion 376 covers only a part of the sample collection media 310. For example, the second portion 376 may cover 50% or less, 40% or less, 30% or less, or 20% or less of the length L310 of the sample collection media 310.

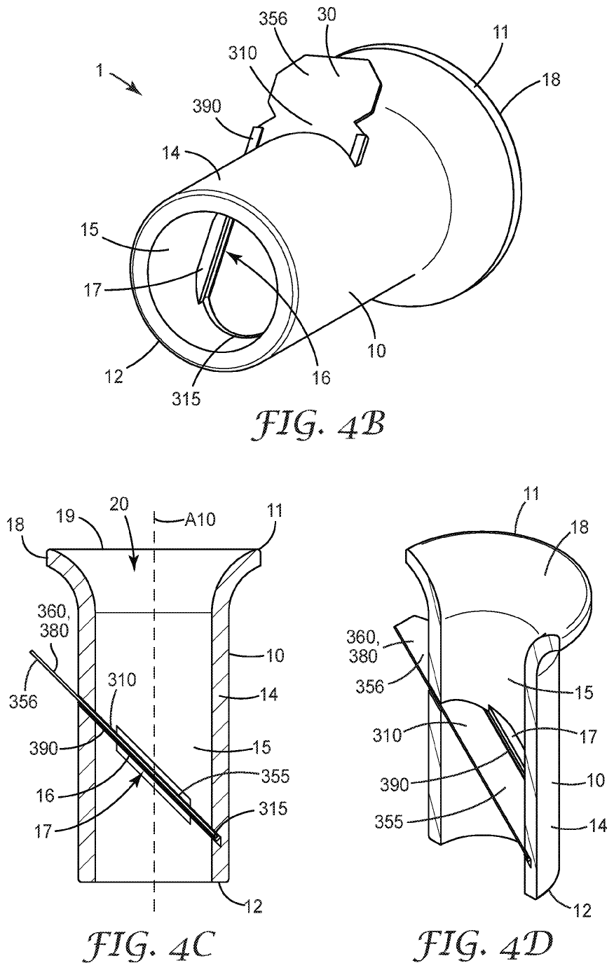

In some embodiments, as shown in FIGS. 3A and 3B, the protected sample collection media 30 is supported by a frame 390. The frame 390 may facilitate insertion of the protected sample collection media 30 into a sample collection device 1. The supportive frame 390 may be disposed along an outer edge 303 of the protective sheath 350 or the sample collection media 310. The supportive frame 390 may be adhered to the protective sheath 350 or the sample collection media 310 by an adhesive.

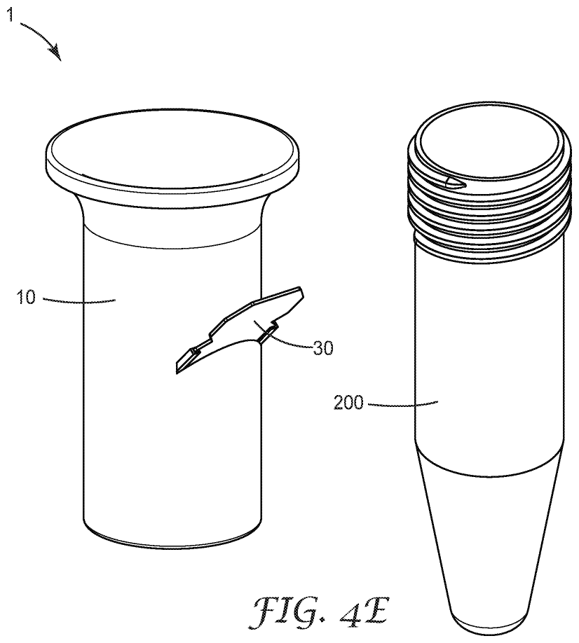

Referring now to FIGS. 4A-4D, the protected sample collection media 30 may be placed (e.g., inserted) within the housing 10 of a sample collection device 1. The sample collection media 310 may be arranged to occlude an airflow path 20 extending through the housing 10.

The exemplary sample collection device 1 has a housing 10 with a tubular wall 14. The tubular wall 14 defines a first end 11 and a second end 12 and an airflow path 20 extending therethrough. The first end 11 may form a mouthpiece 18 with an opening 19 into the airflow path 20. The housing 10 has a longitudinal center axis A10. In the embodiment shown, the airflow path 20 extends along the longitudinal center axis A10 of the housing 10. The protected sample collection media 30 is disposed within the housing 10 such that the sample collection media 310 occludes the airflow path 20. The tubular wall 14 of the housing 10 has an opening 13 for insertion of the protected sample collection media 30.

The opening 13 may be a slot in the tubular wall 14. The slot (opening 13) may be disposed at a non-perpendicular angle relative to the longitudinal axis A10 of the housing 10. The slot may be disposed at a non-perpendicular angle relative to the airflow path 20. In some embodiments, the opening may be formed by a gap between two parts of the tubular wall. The outer edge 303 of the protected sample collection media 30 may either extend along the inside surface 15 of the tubular wall 14 or may extend into a groove 16 in the inside surface 15.

The housing 10 may include a track 17 for supporting one or more edges 303 of the protected sample collection media 30. In the embodiment shown, the housing 10 has two tracks 17 on either side of the interior or the housing 10. The two tracks 17 are angled relative to the longitudinal center axis A10 of the housing 10 and thus the airflow path 20. This increases the surface area of the sample collection media 310 exposed to exhalation airflow. In some embodiments, the two tracks 17 extend inwardly from the inside surface 15 of the housing. The tracks 17 may form a groove 16 along the inside surface 15 of the housing.

In embodiments, where the protected sample collection media 30 is supported by a frame 390, the frame 390 may be inserted into the grooves 16 in the tracks 17 to guide the sample collection media 310 into place.

A user may insert the protected sample collection media 30 into an opening 13 in the sample collection device 1. The second end 316 of the sample collection media 310 and the second portion 352. 372 of the protective sheath 350, 370 form a tab 360, 380 constructed to be gripped by hand. The tab 360, 380 may at least partially extend outside of the sample collection device 1 (e.g., outside of the tubular wall 14). The sample collection device 1 may include various features to facilitate the insertion of the sample collection media, as discussed above.

The user may exhale into the sample collection device 1 and load the sample collection media 310 with a sample of the exhalation airflow to form a loaded porous sample collection media. The user may exhale through the opening 19 in the mouthpiece 18. The housing 10 is constructed so that by exhaling through the opening 19 in the mouthpiece 18, at least a portion of the exhalation airflow passes through the sample collection media 310.

A user may optionally transfer the sample into a sample collection tube 200, shown in FIG. 4E. After loading the sample collection media 310 with a sample, a user may prepare the sample collection media 310 for transfer into a sample collection tube 200 removing the protected sample collection media 30 from the device 1 and removing the first portion 351, 371 of the protective sheath 350, 370, leaving the second portion 352, 372 to cover a part of the sample collection media 310 (e.g., the tab 360, 380).

The first portion 351, 371 of the protective sheath 350, 370 may remain in the opening 13 of the sample collection device 1 as the protected sample collection media 30 is pulled out of the opening 13 by the tab 350, 380. The sample may be transferred by transferring the loaded sample collection media 310 into the sample collection tube 200, for example, by holding the loaded sample collection media 310 by the tab 360, 380 and placing the loaded sample collection media 310 in the sample collection tube 200. The user may further remove the second portion 352, 372 from the tab 360, 380 while placing the sample collection media 310 into the sample collection tube 200. Alternatively, the sample may be transferred by eluting the sample with a suitable eluent. The eluent may be applied through the airflow path 20.

Optionally, a method of using the sample collection media 310 may include applying a liquid to the loaded sample collection media 310. The liquid may be applied in an amount suitable for eluting viruses, pathogens, or other analytes, captured in the porous sample collection media. A suitable amount of liquid may be determined as a ratio of liquid volume to the surface area of the sample collection media 310. For example, the volume of liquid may be in a range from 10 μm/cm² to 400 μm/cm², or from 10 μm/cm² to 250 μm/cm², or from 50 μm/cm² to 150 μm/cm². In some embodiments, the volume of liquid is from 50 μm to 500 μm.

The method may further include vortexing the sample collection tube containing the loaded sample collection media 310.

The protected sample collection media may be provided as part of a sample collection system. The system may include a sample collection device and the protected sample collection media for use with the sample collection device. The system, as shown in FIG. 4E, may further include a sample collection tube into which a sample obtained by using the sample collection device and the protected sample collection media may be transferred.

The protected sample collection media may be provided as part of a kit. The kit may include a sample collection device, the protected sample collection media, and instructions for collecting a sample onto the sample collection media. The instructions may include instructions to: grip the protected sample collection media by the second portion; insert the exposed sample collection media into a sample collection device; exhale along the airflow path to capture a sample in the porous sample collection media; remove the protected sample collection media from the sample collection device; remove the first portion of the protective sheath; and transfer the loaded sample collection media into a sample collection tube or elute the sample from the loaded sample collection media using a liquid.

### EXEMPLARY EMBODIMENTS

The following is a list of exemplary embodiments according to the present disclosure.

Embodiment 1 is a protected sample collection media for use in a sample collection device, the protected sample collection media comprising: a piece of porous sample collection media having a first major side and an opposing second major side; and a protective sheath fully covering the first major side and the second major side. The protective sheath comprises a first portion and a second portion. The first portion is removable without removing the second portion, the second portion forming a tab constructed to be gripped by hand.

Embodiment 2 is the protected sample collection media of embodiment 1 further comprising a supportive frame disposed along the protective sheath or the piece of porous sample collection media. The frame may be adhered to the protective sheath or the piece of porous sample collection media by an adhesive.

Embodiment 3 is the protected sample collection media of embodiment 2, wherein the supportive frame extends along outer edges of the protective sheath or the piece of porous sample collection media.

Embodiment 4 is the protected sample collection media of any one of embodiments 1 to 3, wherein the protective sheath comprises paper, plastic, metal, nonwoven sheet, or a combination thereof. The protective sheath may be made of a porous laminated or coated material, such as plasticized paper, metalized paper, metalized plastic, and the like. The protective sheath may be made of nonwoven material. The nonwoven material may include polylactic acid, polypropylene, or a combination thereof.

Embodiment 5 is the protected sample collection media of any one of embodiments 1 to 4, wherein the protective sheath acts as a pre-filter or screen covering the porous sample collection media during sample taking.

Embodiment 6 is the protected sample collection media of any one of embodiments 1 to 5, wherein the first portion of the protective sheath comprises a peelable first side disposed on the first major side of the piece of porous sample collection media and a peelable second side disposed on the first major side of the piece of porous sample collection media.

Embodiment 7 is the protected sample collection media of any one of embodiments 1 to 4, wherein the first and second portions are integral with one another.

Embodiment 8 is the protected sample collection media of any one of embodiments 1 to 5 and 7, wherein a separation between the first and second portions is marked by notches or perforations.

Embodiment 9 is the protected sample collection media of any one of embodiments 1 to 8, wherein the porous sample collection media comprises nonwoven material.

Embodiment 10 is the protected sample collection media of embodiment 9, wherein the nonwoven material comprises polylactic acid, polypropylene, or a combination thereof.

Embodiment 11 is a sample collection system comprising: the protected sample collection media of any one of the preceding embodiments; and a sample collection device comprising an airflow path extending from a first end to a second end, and an opening constructed to receive the protected sample collection media such that the piece of porous sample collection media occludes the airflow path and the tab at least partially extends outside of the sample collection device.

Embodiment 12 is the sample collection system of embodiment 11, wherein the opening comprises a slot.

Embodiment 13 is the sample collection system of embodiment 12, wherein the slot is disposed at a non-perpendicular angle relative to the airflow path.

Embodiment 14 is the sample collection system of any one of embodiments 11 to 12, wherein the sample collection device comprises a tubular wall defining the first end and the second end, and wherein the first end forms a mouthpiece.

Embodiment 15 is the sample collection system of embodiment 14, wherein the opening is an opening in the tubular wall.

Embodiment 16 is the sample collection system of embodiment 15, wherein the opening is formed between two parts of the tubular wall.

Embodiment 17 is the sample collection system of any one of embodiments 12 to 16, wherein the tubular wall comprises a track on an inside surface of the tubular wall constructed to guide or support the protected sample collection media.

Embodiment 18 is the sample collection system of any one of embodiments 12 to 17, wherein the tubular wall comprises a groove on an inside surface of the tubular wall constructed to guide or support the protected sample collection media.

Embodiment 19 is the sample collection system of any one of embodiments 11 to 18, wherein the sample collection device is constructed for coupling with a sample collection tube via a bayonet coupling, interference fit, snap fit, or threaded coupling.

Embodiment 20 is a kit for collecting a sample, the kit comprising: the sample collection system of any one of embodiments 11 to 19; and instructions to: grip the protected sample collection media by the second portion; insert the exposed sample collection media into the sample collection device; exhale along the airflow path to capture a sample in the porous sample collection media; remove the protected sample collection media from the sample collection device; and remove the first portion of the protective sheath.

Embodiment 21 is the kit of embodiment 20 further comprising instructions to transfer the loaded sample collection media into a sample collection tube. The instructions may further include instructions to remove the second portion of the protective sheath prior to placing the loaded sample collection media in the sample collection tube.

Embodiment 22 is the kit of embodiment 20 or 21 further comprising instructions to elute the sample from the loaded sample collection media using a liquid.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A protected sample collection media for use in a sample collection device, the protected sample collection media comprising:
a piece of porous sample collection media having a first major side and an opposing second major side; and
a protective sheath fully covering the first major side and the second major side, the protective sheath comprising a first portion and a second portion, wherein the first portion is removable without removing the second portion, the second portion forming a tab constructed to be gripped by hand.

2. The protected sample collection media of claim 1 further comprising a supportive frame disposed along the protective sheath.

3. The protected sample collection media of claim 2, wherein the supportive frame extends along outer edges of the protective sheath.

4. The protected sample collection media of claim 1, wherein the protective sheath comprises paper, plastic, metal, nonwoven sheet, or a combination thereof.

5. The protected sample collection media of claim 1, wherein the first portion of the protective sheath comprises a peelable first side disposed on the first major side of the piece of porous sample collection media and a peelable second side disposed on the first major side of the piece of porous sample collection media.

6. The protected sample collection media of claim 1, wherein the first and second portions are integral with one another.

7. The protected sample collection media of claim 1, wherein a separation between the first and second portions is marked by notches or perforations.

8. The protected sample collection media of claims 1, wherein the porous sample collection media comprises nonwoven material.

9. The protected sample collection media of claim 8, wherein the nonwoven material comprises polylactic acid, polypropylene, or a combination thereof.

10. A sample collection system comprising:
the protected sample collection media of any one of the preceding claims; and
a sample collection device comprising an airflow path extending from a first end to a second end, and an opening constructed to receive the protected sample collection media such that the piece of porous sample collection media occludes the airflow path and the tab at least partially extends outside of the sample collection device.

11. The sample collection system of claim 10, wherein the opening comprises a slot.

12. The sample collection system of claim 11, wherein the slot is disposed at a non-perpendicular angle relative to the airflow path.

13. The sample collection system of claim 10, wherein the sample collection device comprises a tubular wall defining the first end and the second end, and wherein the first end forms a mouthpiece.

14. The sample collection system of claim 13, wherein the opening is an opening in the tubular wall.

15. The sample collection system of claim 13, wherein the opening is formed between two parts of the tubular wall.

16. The sample collection system of claim 13, wherein the tubular wall comprises a track on an inside surface of the tubular wall constructed to guide or support the protected sample collection media.

17. The sample collection system of claim 10, wherein the sample collection device is constructed for coupling with a sample collection tube via a bayonet coupling, interference fit, snap fit, or threaded coupling.

18. A kit for collecting a sample, the kit comprising:
the sample collection system of claims 10; and instructions to:
grip the protected sample collection media by the second portion;
insert the exposed sample collection media into the sample collection device;
exhale along the airflow path to capture a sample in the porous sample collection media;
remove the protected sample collection media from the sample collection device; and
remove the first portion of the protective sheath.

19. The kit of claim 18 further comprising instructions to transfer the loaded sample collection media into a sample collection tube.

20. The kit of claim 18 further comprising instructions to elute the sample from the loaded sample collection media using a liquid.
